(19) United States
(12) Patent Application Publication
(10) Pub. No.: US 2015/0080817 A1
(43) Pub. Date: Mar. 19, 2015

(54) Ostomywear by cathie

(71) Applicant: Cathrine Marie Beliveau, Wakefield, MA (US)

(72) Inventor: Cathrine Marie Beliveau, Wakefield, MA (US)

(21) Appl. No.: 13/987,899

(22) Filed: Sep. 13, 2013

(51) Int. Cl. A61F 5/449

(52) U.S. Cl. CPC A61F 5/449
USPC 604/345

(57) ABSTRACT

Ostomywear by Cathie is a fabric covering, used as a protective barrier over one's colostomy bag. A colostomy bag is made from a plastic, similar to a zip and seal sandwich bag, which is attached to the body by a wax and band aid type skin barrier, adhered to the skin over one's colostomy stoma. The ostomywear is made from designed cotton and blended fabrics, a decorative or plain outside fabric and a lining fabric. Between the outside fabric and the lining is a fabric of high strength, light weight material known as KEVLAR™.

My Ostomywear by Cathie is slipped over one's colostomy bag and attached with a hook and loop fastener. Worn under the clothing, one will feel confident to go about their day. The benefit of Ostomywear by Cathie, is protection of the colostomy bag and stoma from tears and trauma, as well as being fashionable.

### BACKGROUND OF INVENTION

I am a survivor of colon cancer, and I have to wear a colostomy bag. The colostomy bag is made from plastic similar to a zip and seal bag. It is not very attractive, as well as not very strong. I love to sew, and I have made pretty covers for myself to go over the bag.

I am married and have two sons. I work in a hospital as a nursing assistant, as well as grooming dogs. I work outside in my gardens, and I do everything I have always done before cancer. One thing that has always worried me is, what would happen if my colostomy site got hit or traumatized and the bag ripped?

After a lot of thought, KEVLAR™ came to mind. It is used in gloves, helmets, bullet proof vests, among other products. Why not try this high strength material and incorporate it into my colostomy bag covers? I had looked on the internet and found a company named National Nonwovens in Easthampton, Mass., and they had sent a sample yard of the KEVLAR™ fabric to me.

### BRIEF SUMMARY OF THE INVENTION

I have incorporated the KEVLAR™ fabric into the design of my Ostomywear by Cathie colostomy bag covers, and after a few adjustments in my pattern, it worked out well. I wear it while I'm working and it's pretty, as well as a great barrier to tears and punctures. I wear it under my clothes and feel confident. When sewn with a more masculine favored design fabric, Ostomywear by Cathie will be a great colostomy bag protection for construction workers, law enforcement officers, sports men and women, anyone whom has to wear a colostomy bag and has to go out and work.

### BRIEF DESCRIPTION OF THE SEVERAL VIEWS OF THE DRAWINGS

Page #1.

FIG. 1. View of paper pattern pinned to outside fabric. 22.5 inches long by 7.5 inches wide. 3.5 outside (head) curve. 3.5 inches deep inside (tail) curve.

FIG. 2. View of the paper pattern pinned to KEVLAR™ fabric, 11.25 inches long by 7.25 inches wide. 3.25 outside curve.

Page #2.

FIG. 3. View of the right side fabric cut out.

FIG. 4. View of the lining fabric with one side of hook and loop fastener sewn on, and with isometric view of the KEVLAR™ fabric sewn onto opposite side of lining fabric.

Page #3.

FIG. 5. View of the lining fabric pinned to the outside fabric, right sides together, with isometric view of hook and loop fastener on right side of lining, with the KEVLAR™ on the opposite side, ready to be sewn together. Take out pins and cut six small notches, turn fabrics so the wrong sides are together, and with a hot iron steam press the tail curve into shape.

FIG. 6. View of the folding of outer fabric, right sides together, head to tail, and fold lining fabric, right sides together, head to tail.

FIG. 7. View of the fabrics pinned together, leaving the fold and a two inch opening for turning right side out. Sew, turn right side out. Sew the opening closed, press with the steam iron.

Page #4.

FIG. 8. View of the Ostomywear by Cathie with colostomy bag, and partial isometric view of colostomy bag inside, as well as isometric view of hook and loop fastener on skin barrier which fastens the colostomy bag to the Ostomywear by Cathie.

FIG. 9. View of Ostomywear by Cathie with isometric view of the colostomy bag.

Page #5.

FIG. 10. View of person with colostomy stoma.

FIG. 11. View of person wearing colostomy bag over stoma.

FIG. 12. View of person wearing Ostomywear by Cathie, with the KEVLAR™ protection, over the colostomy bag.

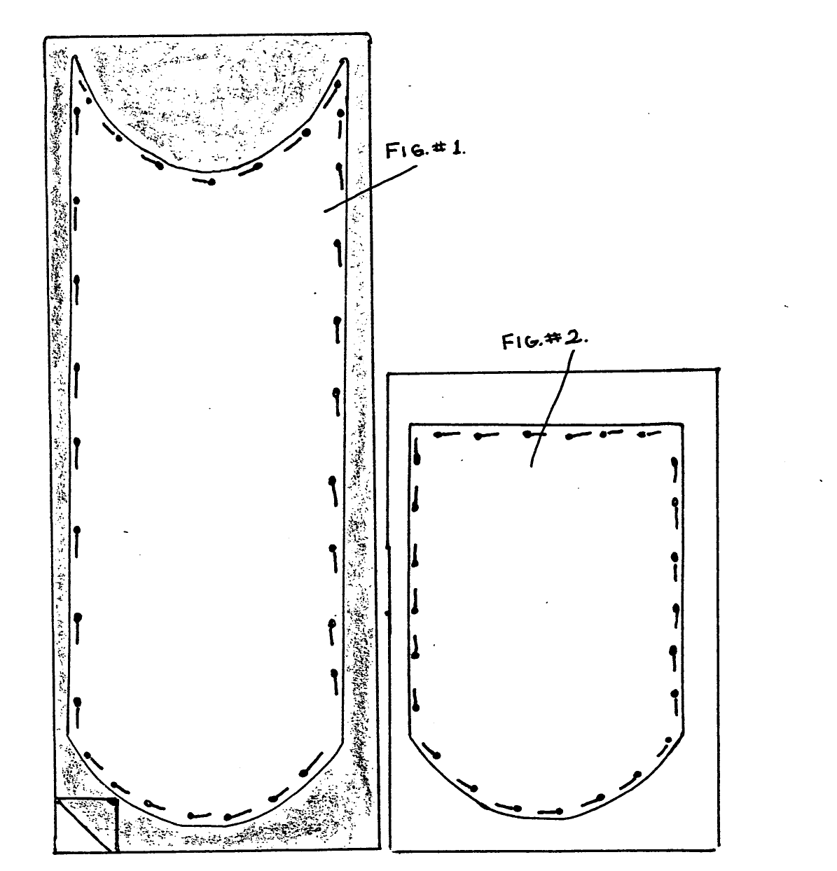

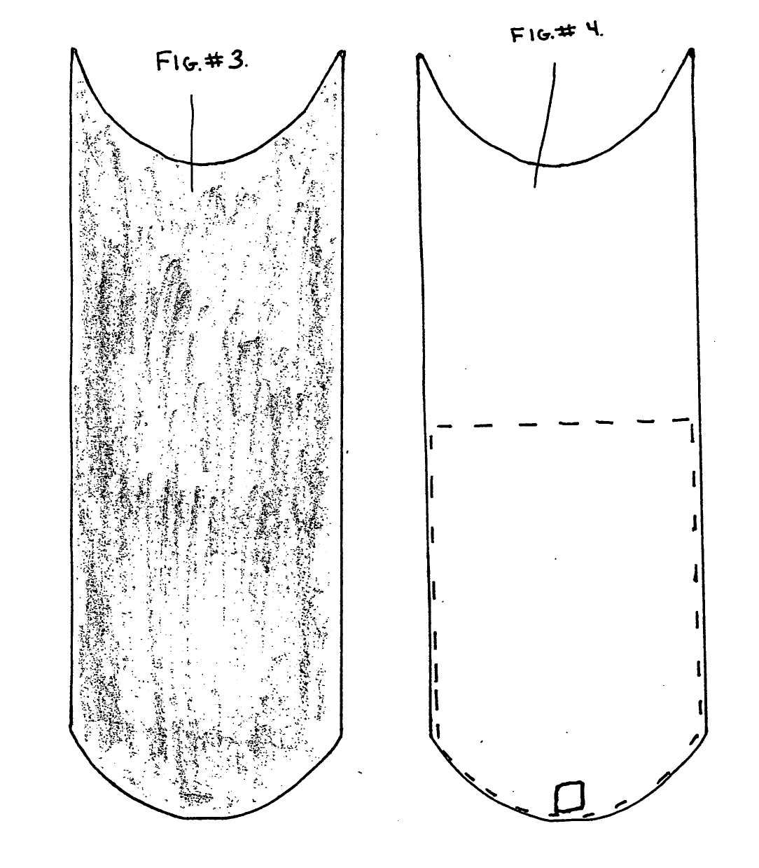

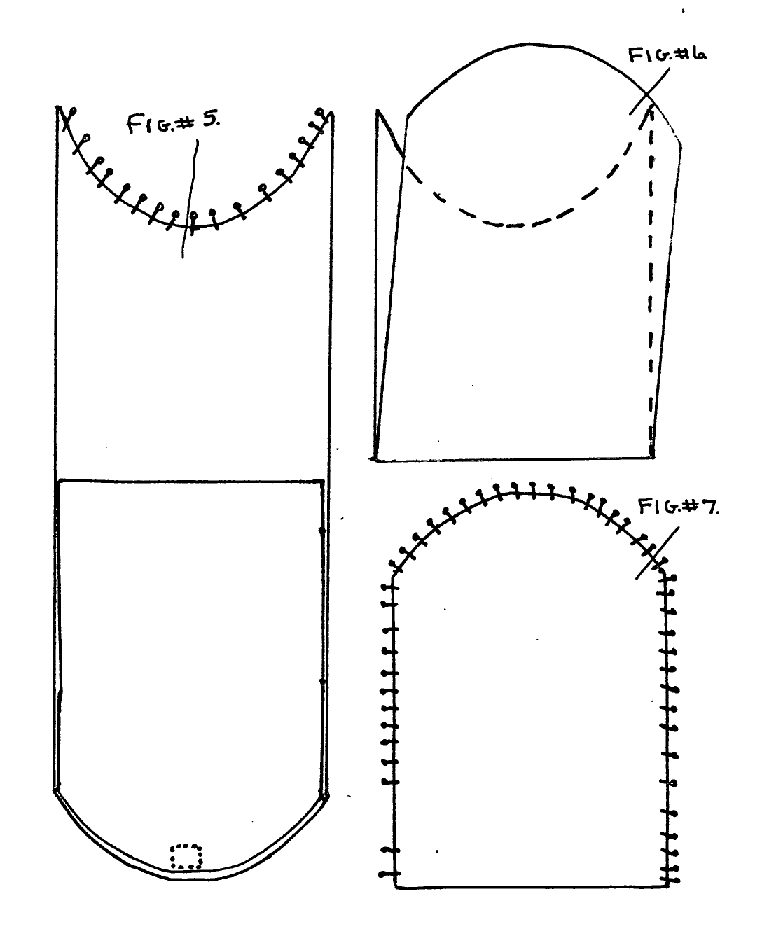

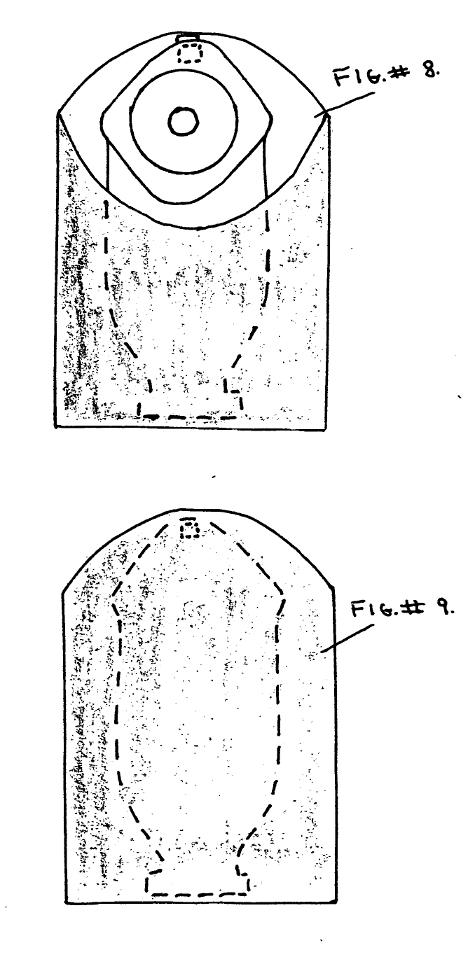

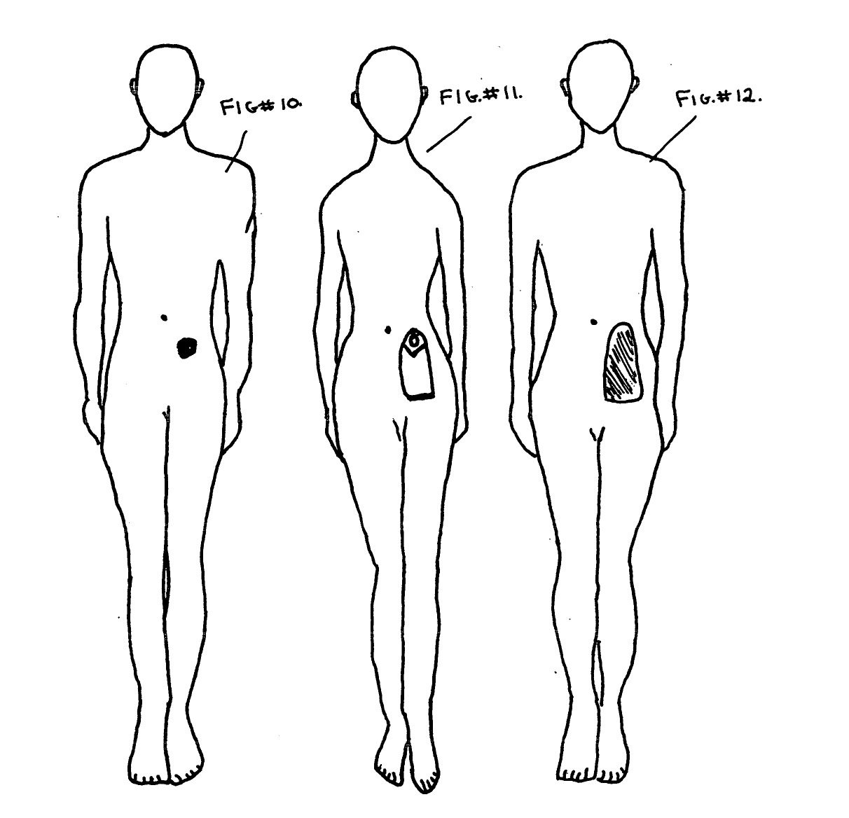

I claim:

1. My invention is going to help colostomy patients feel secure and confident while at work, during any activity, when wearing Ostomywear by Cathie over their colostomy bag.
(a.) The cotton, cotton blends, and nylon fabrics of Ostomywear by Cathie, covering the colostomy bag, is cool and soft against the skin.
(b.) The said KEVLAR™ fabric sewn in between the outside fabrics, when worn over the colostomy bag, will protect the colostomy bag from trauma.
(c.) With a one inch square of sewn in hook and loop fastener to the lining of the ostomywear, and the opposite side one inch square of sticky back hook and loop fastener adhered to the top of the skin barrier of the colostomy bag, slip the colostomy bag into the Ostomywear by Cathie, the colostomy bag and stoma will be protected and you will look great.
(d.) Ostomywear by Cathie is washable and dryable.
